(19) United States
(12) Patent Application Publication
(10) Pub. No.: US 2026/0224170 A1
(43) Pub. Date: Aug. 6, 2026

(54) BRADYCARDIA PREDICTION AND INTERRUPTION

(71) Applicant: THE UAB RESEARCH FOUNDATION, BIRMINGHAM, AL (US)

(72) Inventors: Colm TRAVERS, Homewood, AL (US); Arie NAKHMANI, Mountain Brook, AL (US); Namasivayam AMBALAVANAN, Vestavia Hills, AL (US); Waldemar Alberto CARLO, Vestavia Hills, AL (US)

(21) Appl. No.: 19/157,425

(22) PCT Filed: Feb. 19, 2024
(86) PCT No.: PCT/US2024/016352

§ 371 Date: Aug. 18, 2025

Related U.S. Application Data
(60) Provisional application No. 63/485,970, filed on Feb. 20, 2023.

(51) Int. Cl. A61B 5/00; A61B 5/024; A61H 23/00; A61H 31/00; G16H 20/30

(52) U.S. Cl. CPC A61B 5/7275; A61B 5/02405; A61B 5/4836; A61B 5/746; A61B 5/747; A61H 23/00; A61H 31/006; G16H 20/30

(57) ABSTRACT

The present disclosure relates to systems and methods for bradycardia prediction and interruption based in part on biomedical signal information received from a sensor positioned on a person. The system can include a computing device in data communication with the sensor. The computing device can receive biomedical signal information from the sensor, including at least heart rate data. The computing device can evaluate a sequence of heart rate values over an interval of time based at least in part on the heart rate data and forecast a predicted heart rate value for a subsequent time. In response to the predicted heart rate value being less than a predetermined threshold value, the computing device can generate an intervention signal to prompt an intervention. The computing device can transmit the intervention signal to an intervention apparatus in communication with the computing device to cause at least a vibration to simulate the person.

### CROSS-REFERENCE TO RELATED APPLICATIONS

This application claims the benefit of and priority to U.S. Provisional Application having Ser. No. 63/485,970, having the title “BRADYCARDIA PREDICTION AND INTERRUPTION,” filed on Feb. 20, 2023, the disclosure of which is incorporated herein by reference in its entirety.

### BACKGROUND

Biomedical signals are utilized in clinical practice to determine and monitor the medical condition and alert caregivers to a patient deterioration. Apnea of prematurity is common in very preterm infants and leads to frequent episodes of severe slowing of the heart rate (bradycardia) and drops in blood oxygen levels (hypoxemia). Apnea of prematurity is associated with neurodevelopmental impairment and the development of chronic lung disease. Continuous monitoring of these events is laborious, bringing high costs to healthcare and impeding accurate diagnosis and early intervention.

### BRIEF DESCRIPTION OF THE DRAWINGS

The foregoing will be apparent from the following more particular description of example embodiments of the present disclosure, as illustrated in the accompanying drawings in which like reference characters refer to the same parts throughout the different views. The drawings are not necessarily to scale, emphasis instead being placed upon illustrating embodiments of the present disclosure.

FIG. 1 illustrates an example of the bradycardia prediction and interruption system according to various embodiments of the present disclosure.

FIG. 2 illustrates an example method for bradycardia prediction and interruption according to various embodiments of the present disclosure.

FIG. 3 illustrates an example deep learning architecture for using simple recurrent neural network (RNN) autoencoder prediction model according to various embodiments of the present disclosure.

FIG. 4 illustrates an example deep learning architecture using a transformer prediction model according to various embodiments of the present disclosure.

FIG. 5 illustrates a graphic example of a Boolean function used to describe the predictor evaluation process according to various embodiments of the present disclosure.

FIG. 6 illustrates a graphic comparison of the performance of several prediction models according to various embodiments of the present disclosure.

FIG. 7 illustrates a graphic example of calculated values of several prediction models according to various embodiments of the present disclosure.

### DETAILED DESCRIPTION

The present disclosure relates to a bradycardia prediction and interruption system for monitoring the biomedical signals of a person for bradycardia episodes to prompt interruption of an episode by vibrotactile stimulation. Bradycardia episodes, frequently associated with apnea, are common in preterm infants and associated with bronchopulmonary dysplasia and neurodevelopmental impairment. Prediction of a bradycardia event before occurrence allows for intervention to reduce the frequency or severity of these episodes, potentially improving outcomes. The bradycardia prediction and interruption system provides an automated system and method for signal prediction to allow more accurate and early diagnosis or prognosis. The bradycardia prediction and interruption system can predict severe apnea-bradycardia events before an occurrence to prompt intervention to reduce the frequency or severity of these episodes.

Monitoring of biomedical signals, such as heart rate, respiratory rate, and oxygen saturation monitoring have become a ubiquitous part of neonatal assessment. Apnea of prematurity is common in very preterm infants and leads to frequent episodes of severe slowing of the heart rate (bradycardia) and drops in blood oxygen levels (hypoxemia). Apnea of prematurity is associated with neurodevelopmental impairment and the development of chronic lung disease. In an embodiment, the bradycardia prediction and interruption system can utilize continuously measured vital signs of infants, such as preterm infants recorded in neonatal intensive care units, to predict bradycardia events and to interrupt the event with vibrotactile stimulation.

Physical stimulation is recognized as an important means to abort apneic episodes and encourage resumption of breathing in preterm infants. Early prediction is essential for reducing the risk of impaired cognitive function in the long term. Vibrotactile stimulation can interrupt episodes of bradycardia due to apnea in preterm infants but vibrotactile stimulators are not currently used in routine clinical practice. The bradycardia prediction and interruption system described herein can predict episodes of bradycardia prior to occurrence and provide vibrotactile stimulation using a closed-loop system to interrupt or disrupt these episodes. In some examples, the system can also be connected to an alarm or provide notifications to prompt intervention by a caregiver. While the example system and method described herein relates to infants, prediction of changes in biomedical signals, such as vital signs, is useful for many clinical applications, as such the system can be adapted and relied on for prediction and intervention based on monitoring of biomedical signals in other patients and/or environments.

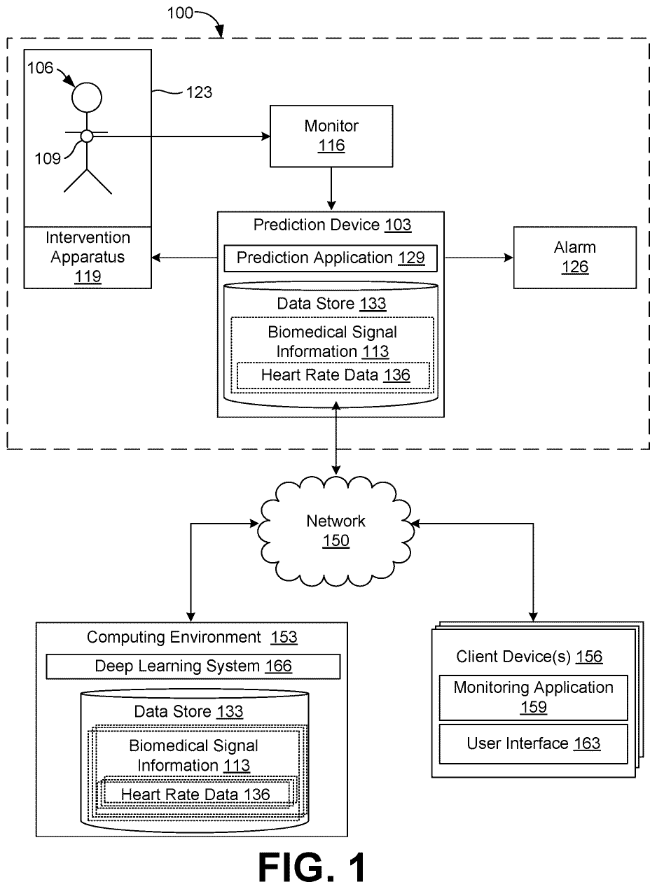

FIG. 1 illustrates an example of a bradycardia prediction and interruption system 100 according to various embodiments of the present disclosure. According to various embodiments, the bradycardia prediction and interruption system 100 can include a prediction device 103 in an environment, such as a hospital unit, where a person 106 is being monitored by at least one sensor 109. In some examples, the person 106 being monitored may be an infant in a neonatal intensive care unit (NICU). In some examples, the person 106 may be a pediatric or adult patient in another environment. The at least one sensor 109 is adapted to be positioned on the person 106 to collect biomedical information 113. The biomedical information can include heart rate, respiratory rate, blood oxygen saturation, as well as, other vital signs or measurable biomedical information about the person 106. In some examples, the at least one sensor 109 can include one or more electrodes placed on the chest of a person 106 for electrocardiogram (ECG or EKG), blood oxygen saturation and/or pulse oximetry monitoring, which may be connected to a monitor 116. In some examples, the monitor 116 may be in place to display real-time biomedical signal information 113.

The prediction device 103 can be adapted to receive biomedical signal information 113 from the at least one sensor 109 positioned on the body of the person 106. In some examples, biomedical signal information 113 from the at least one sensor 109 can be received via the monitor 116 or other systems in data communication with the prediction device 103. In some examples, the prediction device 103 can have a direct connection to the at least one sensor 106. The bradycardia prediction and interruption system 100 can also include an intervention apparatus 119 in communication with the prediction device 103. In some examples, intervention apparatus 119 can be a vibrotactile device positioned on the person 106 or coupled to a bed 123 on which the person 106 is resting. In an example, the intervention apparatus 119 can be a wearable vibrotactile device, worn by the person 106, that vibrates in response to receiving an intervention signal from the prediction device 103. In another example, the intervention apparatus 119 can be a vibrotactile device positioned in or under or otherwise coupled to the bed 123 of the person 106. For example, the bed 123 can be a patient bed, crib, bassinet, or incubator. In some examples, the bradycardia prediction and interruption system 100 can also include an alarm 126 in communication with the prediction device 103. The alarm 126 can be configured to produce an audible or visual alert, in response to receiving an intervention signal or additional signal from the prediction device 103.

The prediction device 103 comprises at least one computing device comprising a processor and a memory. A prediction application 129 may be executed by the processor in the prediction device 103 according to various embodiments. Also, various data is stored in a data store 133 that is accessible to the prediction device 103. The data store 133 may be representative of a plurality of data stores 133 as can be appreciated. The data stored in the data store 133 for example, is associated with the operation of the various applications and/or functional entities described below. For example, the biomedical signal information 113 received from the sensors 109 and/or heart rate data 136 can be stored in the data store 133.

The prediction application 129 can receive the biomedical signal information, including the measured heart rate and use an algorithm of the prediction application 129 to predict bradycardia (low heart rate) episodes before they occur. The prediction algorithm can be a statistical algorithm, as will be described in more detail herein. The prediction algorithm can be a linear algorithm, a linear regression algorithm, and/or a finite difference algorithm to predict bradycardia episodes before they occur. In an example, the heart rate data used by the prediction algorithm can potentially be received as a signal or data from any heart rate monitor that is connected to the person or patient. The heart rate signal can be processed by the processor embedded in the device.

The prediction application 129 causes the prediction device 103 to at least receive biomedical signal information 113 from the at least one sensor 109 positioned on the person 106. The biomedical signal information 113 can include at least heart rate data 136 but can also include additional data related to the person 106 or the vital signs of the person 106. The prediction application 129 can evaluate a sequence of heart rate values over an interval of time based at least in part on the heart rate data 136 received and can forecast a predicted heart rate value for a subsequent time. For example, the system 100 can predict a bradycardia episode about 1-10 seconds before the event allowing an interval of response to an episode. In response to the predicted heart rate value being less than a predetermined threshold value, generate an intervention signal to prompt intervention. For example, the predetermined threshold value can be 80 bpm, 100 bpm, or another heart rate relevant to the health condition of the person. The prediction application 129 can transmit the intervention signal to an intervention apparatus in communication with the prediction device, where the intervention apparatus is adapted to receive the intervention signal from the prediction device and cause at least a vibration to simulate the person.

In an example, the prediction device 103 can monitor simple outputs prior to using the novel predictive algorithm of the predictive application. For example, the prediction device 103 can use the open-source library of ProtoCentral ECG/Respiration under the MIT License and open hardware to monitor simple outputs. If bradycardia is predicted or detected by the algorithm of the prediction application, a vibrotactile stimulator connected to the patient can be activated to interrupt and disrupt the bradycardia episode. In various examples, the device can be isolated from the main power. Both the embedded board and heart rate monitor can be open-source hardware, i.e., their schematic diagrams are openly available. For testing purposes, additional Micro NeoLead ECG electrodes or Red Dot electrodes that are connected to a Philips monitor cable as typically used and connected in routine care in the neonatal intensive care unit. The prediction device 103 can be interfaced directly to the Philips cable rather than the Philips monitor so that there will be no difference in the application or connection of electrodes compared to routine clinical practice.

The prediction device 103 can be coupled, wired or wirelessly, to the sensors 109 directly or via a monitor, the intervention apparatus 119, such as a vibrotactile device, and optionally an alarm 126 that can provide an audible or visual alert, as described above. In addition, the prediction device 103 can include a communication interface (not shown) that allows the prediction device 103 to communicatively couple with other communication devices. The communication interfaces may include one or more wireless connection(s) such as, e.g., Bluetooth or other radio frequency (RF) connection and/or one or more wired connection(s). In some examples, the prediction device 103 further can be data communication with one or more computing devices over a network 150. For example, as shown in FIG. 1 the prediction device 103 can be in data communication with a computing environment 153 and one or more client devices 156.

In some examples, the prediction device 103 can be in data communication with a client device 156. For example, the prediction device 103 can be configured to send a notification signal to the client device 156 in response to the prediction application 129 forecasting a predicted heart rate value that is less than a predetermined threshold value. The client device 156 (e.g., mobile device, computer, etc.) can include a monitoring application 159 and a user interface 163 to interface with the user. For example, the prediction device 103 can transmit the notification signal to the client device 156 to alert the user via the monitoring application 159 that intervention is needed or has been initiated by the intervention apparatus 119. In some examples, the real-time monitoring data may be transmitted from the prediction device 103.

A computing environment 153 may comprise, for example, a server computer or any other system providing computing capability. Alternatively, the computing environment 153 may employ a plurality of computing devices that may be arranged, for example, in one or more server banks or computer banks or other arrangements. Such computing devices may be located in a single installation or may be distributed among many different geographical locations. For example, the computing environment 153 may include a plurality of computing devices that together may comprise a hosted computing resource, a grid computing resource and/or any other distributed computing arrangement. In some cases, the computing environment 153 may correspond to an elastic computing resource where the allotted capacity of processing, network, storage, or other computing-related resources may vary over time.

Various applications and/or other functionality may be executed in the computing environment 153 according to various embodiments. Also, various data is stored in a data store 133 that is accessible to the computing environment 153. The data store 133 may be representative of a plurality of data stores 133 as can be appreciated. The data stored in the data store 133, for example, is associated with the operation of the various applications and/or functional entities described below. For example, the biomedical signal information 113 and the heart rate data 136, received by the prediction device 103, can also be stored in a data store 133 in the computing environment 153.

The components executed on the computing environment 153, for example, include list of applications, and other applications, services, processes, systems, engines, or functionality not discussed in detail herein. In some examples, the prediction application 103 comprises a statistical algorithm to predict bradycardia in a person 106 being monitored. The statistical algorithm can be at least one of: Last Observation Carried Forward (LOCF), Linear Regression, Linear, and Finite Difference predictors, as described further herein. In some examples, the prediction device 103 can further communicate data to a computing environment and may utilize a deep learning application 166 to predict bradycardia in a person 106 being monitored. The deep learning application 166 can include a deep learning prediction architecture, such as SimpleRNN, Long Short-Term Memory (LSTM), Bidirectional LSTM (Bi-LSTM), Variational AutoEncoder (VAE), Gated Recurrent Units (GRU), and Transformers, as described further herein.

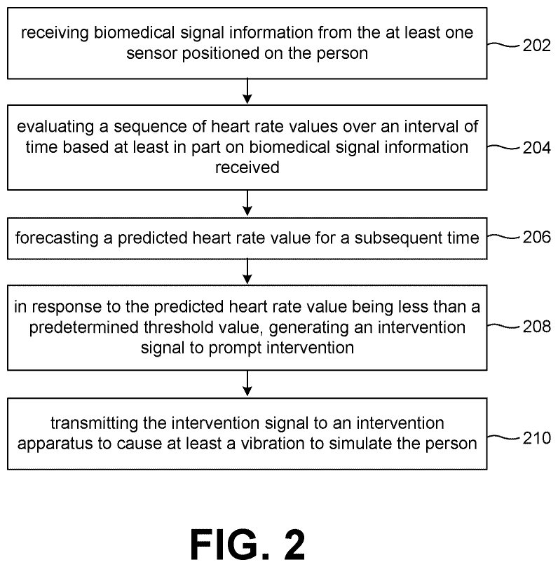

In FIG. 2, an example method for predicting and interrupting bradycardia episodes in a person is shown, according to various embodiments of the present disclosure. The method can be implemented via at least one computing device, such as the prediction device 103, in data communication with at least one sensor 109 positioned on a person 106 being monitored.

At box 202, the method comprises receiving biomedical signal information 113 comprising at least heart rate data 136 of the person 109 being monitored. The biomedical signal information 113 can be received directly or indirectly from at least one sensor 109 positioned on the body of the person 106. The biomedical signal information 113 can be received in real-time via a wired or wireless connection. The biomedical signal information 113 can also include pulse oximetry data, respiration rate data, or other vital sign information of the person 106.

At box 204, the method comprises evaluating a sequence of heart rate values over an interval of time based at least in part on the heart rate data received. The sequence of heart rate values can include the data for the present and recent past data. In some examples, the sequence of heart rate values can be the present heart rate value h(t) and the heart rate at three previous intervals (h(t-1), h(t-2), and h(t-3)).

At box 206, the method comprises forecasting a predicted heart rate value for a subsequent time. The prediction application 129 can utilize a statistical algorithm to predict a bradycardia episode. For example, the statistical algorithm can be at least one of a linear regression algorithm, a predictor based on finite differences, and/or a linear equation. In an example, the prediction application can use a linear regression algorithm to calculate the linear combination of the previous input features with linear least squares error fit of a straight-line model to extrapolate a heart rate for a future time at the next interval. For example, the linear regression predictor of the prediction application uses four samples from the past and present (h(t), h(t-1), h(t-2), and h(t-3)) to fit a straight-line model and extrapolate it for t+T time moment, where T=5. In another example, the prediction application can use a predictor based on finite differences. The assumption for this predictor is that the approximation of a second order derivative is a constant. The predictor uses four signal samples from the last four time steps h(t), h(t-1), h(t-2), and h(t-3) to predict the 5th step. In an alternate example, the prediction application can use a linear predictor that uses the past two samples h(t) and h(t-1) to predict the fifth time step, assuming that the slope of a signal will stay constant.

At box 208, the method comprises in response to the predicted heart rate value being less than a predetermined threshold value, generating an intervention signal to prompt intervention. For example, the predetermined threshold value can be 80 bpm, 100 bpm, or another heart rate relevant to the health condition of the person.

At box 210, the method comprises transmitting the intervention signal to an intervention apparatus to cause at least a vibration to simulate the person. The prediction application 129 can transmit the intervention signal to an intervention apparatus 119 in communication with the prediction device 103, where the intervention apparatus 119 is adapted to receive the intervention signal from the at prediction device 103 and cause at least a vibration to simulate the person 106. In some examples, the intervention signal or an additional signal comprise an alert signal transmitted to an alarm 126 and/or a notification signal transmitted to a client device 156.

### Example

Prediction of changes in biomedical signals, such as vital signs, is useful for many clinical applications. Several signal prediction (forecasting) tools were developed, but their evaluation and applicability to a specific clinical use is context dependent. In this work, a novel method for evaluation and comparison of vital sign predictors for intervention is proposed for clinical studies. Specifically, nine deep learning and statistics based predictive models for multi-step prediction of bradycardia events in preterm infants are studied and compared, but the proposed method could be applied to other biomedical signals. The results on testing sets with several days of vital sign recordings show that simple statistical predictors could outperform state-of-the-art deep learning architectures for low-dimensional signals.

Biomedical signals are utilized in clinical practice to determine and monitor the medical condition and alert caregivers to a patient deterioration. Continuous monitoring of these events is laborious, bringing high costs to healthcare and impeding accurate diagnosis and early intervention. Automated methods or signal prediction may allow more accurate and early diagnosis or prognosis. To choose the best predictor for a task, there is a need to evaluate its diagnostic accuracy. The available evaluation methods do not always adequately address this need. In this work, one example is outlined where no appropriate tools exist that would allow meaningful comparison of methods for multi-step signal prediction. This example relates to prediction of continuously measured vital signs in preterm infants recorded in neonatal intensive care units. Note that the preterm infants included in the study were breathing spontaneously without respiratory support.

Heart rate, respiratory rate, and oxygen saturation monitoring have become a ubiquitous part of neonatal assessment. Apnea of prematurity is common in very preterm infants and leads to frequent episodes of severe slowing of the heart rate (bradycardia) and drops in blood oxygen levels (hypoxemia). Apnea of prematurity is associated with neurodevelopmental impairment and the development of chronic lung disease. Physical stimulation is recognized as an important means to abort apneic episodes and encourage resumption of breathing in preterm infants. Prophylactic cutaneous stimulation may re-duce episodes of apnea compared with routine care, but has only been tested in relatively small clinical trials using physical touch and a variety of kinesthetic devices. However, treating episodes of apnea with bradycardia with vibrotactile devices after detection may have limited efficacy compared with routine care. Prediction of severe apnea-bradycardia events before they occur may allow development of interventions to reduce the frequency or severity of these episodes, potentially improving outcomes. Early prediction is essential for reducing the risk of impaired cognitive function in the long term. Unfortunately, physiological signals such as heart rate are complex with continuous change of amplitudes and exhibit non-stationary and chaotic behaviors.

The accurate prediction of abnormalities/events from biomedical signals is clinically highly relevant and important for saving human lives and improving clinical management decisions. Following are some example of clinical events where precise prediction is required: (1) apneic events, (2) stroke development, (3) epileptic seizures.

Many machine learning (ML) approaches have been explored in the literature for forecasting apneic events of preterm infants and adults. However, relatively few research studies focused on prediction of apneic events using recurrent neural networks (RNN) and RNN has not been used to predict apneic or bradycardic events in preterm infants.

Several signal prediction (forecasting) tools were developed, but their evaluation and applicability to a specific clinical use is context dependent. In this work, a method for evaluation and comparison of vital sign predictors for intervention based clinical studies. Specifically, nine deep learning and statistics based predictive models for multi-step prediction of bradycardia events in preterm infants are studied and compared, but the method could be applied to other biomedical signals. The results on testing sets with several days of vital sign recordings show that simple statistical predictors could outperform state-of-the-art deep learning architectures for low-dimensional signals.

This research study discusses how deep learning and statistics based predictive models can forecast complicated and clinically significant events. Also, these predictive models are compared using the novel predictor evaluation metrics developed in this paper. In an example shown, the evaluation approach with bradycardia event predictions, but prediction of other clinically significant events can be conducted in a similar manner.

### Problem Formulation

Premature neonates tend to have respiratory pauses and instability of the heart rate due to immature autonomous control mechanisms. They are monitored in the intensive care unit 24/7 using multiple sensors to measure electrical heart activity (ECG), blood oxygen saturation, and breathing rate.

The heart rate function of time is denoted by h(t), t=1, 2, . . . , N, where N is the total number of samples in a single recording (typically 24 hours long). A bradycardia event is defined when the heart rate signal drops below 100 beats per minute for more than 10 seconds. To prompt intervention, such events should be predicted at least T seconds (using T-steps prediction) before they happen. It is assumed that a window of w seconds, of data preceding the event, will have sufficient number of features to predict the event. The goal is to find a predictor that will reliably forecast almost all bradycardia events without producing too many false alarms. Moreover, a predictor that signals an event within the time of the event was not penalized or favored.

### Data Sets

The data sets of neonatal heart rate recordings were obtained in a study approved by the UAB IRB. No demographics or any other additional information on patients were used for prediction. The bradycardic events were detected automatically and stored using in-house Python code. The 67 days of heart rate recordings from 6 different patients that contain 2,196 bradycardia events were combined to create training and validation sets for deep learning (DL) based predictors. Details of the data sets are shown in Table 1. Regardless of the actual sampling rate of the sensors, the value of the heart rate is reported once a second by Philips IntelliVue MP70 monitor (typically, averaged on 8 seconds of a raw signal). This makes the sampling rate of Fs=1 Hz, which equates the number of samples per given signal and the number of recorded seconds (total recording time in seconds).

TABLE 1
Training and validation sets for DL-based predictors
from six infants (P3, P5, P6, P8, P11 and P13). The
Data Sample Length column represents total number of
recorded seconds. The Bradycardia Events column gives
total number of bradycardia events per recording.

| Patient Code | Data Samples Length | #Bradycardia Events |
|---|---|---|
| P3 | 3,365,313 | 235 |
| P5 | 172,083 | 124 |
| P6 | 257,106 | 245 |
| P8 | 302,100 | 413 |
| P11 | 919,775 | 823 |
| P13 | 517,486 | 356 |
| Total | 5,533,863 | 2,196 |

A set of 500 day recordings from 115 neonates was used for testing.

### DL-Based Predictors:

Nine predictors (five DL-based and four statistics-based) for forecasting the heart rate signal five time steps (seconds) ahead were studied and compared.

For training purposes, only the days with high number of events per day were selected to enrich the sets with relatively frequent bradycardia events and to better balance the training and validation data sets. The training and validation split ratio was selected to be 80 to 20. Each DL-based predictor was trained on 4,426,760 seconds and validated on 1,106,689 using a sliding window of length w=60 seconds to predict T=5 seconds ahead.

For any particular time t, the heart rate values in the interval h(t-w), . . . , h(t) were utilized to predict the value of h(t+T). This predicted value is denoted by h(t). Since the signal is sampled once per a second, the prediction could be achieved iteratively by repeating T times 1-step prediction.

The deep learning architectures such as SimpleRNN, Long Short-Term Memory (LSTM), Bidirectional LSTM (Bi-LSTM), Variational AutoEncoder (VAE), Gated Recurrent Units (GRU), and Transformers are state-of-the-art techniques in time series prediction domain. In this work, the performance of these architectures on bradycardia events prediction are compared.

### SimpleRNN Autoencoder Predictor

The Autoencoder is a type of artificial neural network architecture that learns a compressed representation of input data. It has two main components: Encoder and Decoder. The Encoder module reads the provided input sequence step by step and represents the whole sequence as a fixed-length vector of encoded features (or compressed data). The Decoder module takes this fixed-length vector as an input and outputs the decoded data. In other words, the Decoder module decompresses the data. The feature extraction capabilities of the autoencoder architecture and the forecasting abilities on time series of RNN (and its variants) can be integrated to achieve the best predictive models. The Encoder-decoder architecture with RNNs has been employed to solve a variety of sequence prediction tasks.

SimpleRNN is a type of fully connected RNN that uses an internal memory structure to remember the information of previous inputs. It does not have gate mechanisms like GRU and LSTM. Thus, the SimpleRNN designs do not preserve long-term dependencies over many time steps, and they are sensitive to vanishing and exploding gradients. The mathematical representation of SimpleRNN is given by:

$${H}_{t} = Sigm oid ( {W}_{H} {X}_{t} + {U}_{H} {H}_{t - 1} + {b}_{H} ) , \qquad ( 1 )$$

where W_{H} and U_{H} are weight matrices, Xₜ represents the input vector, and by represents the bias parameter. Hₜ is the new state at the current time step t, and Hₜ₋₁ is the previous state at the t-1 time step.

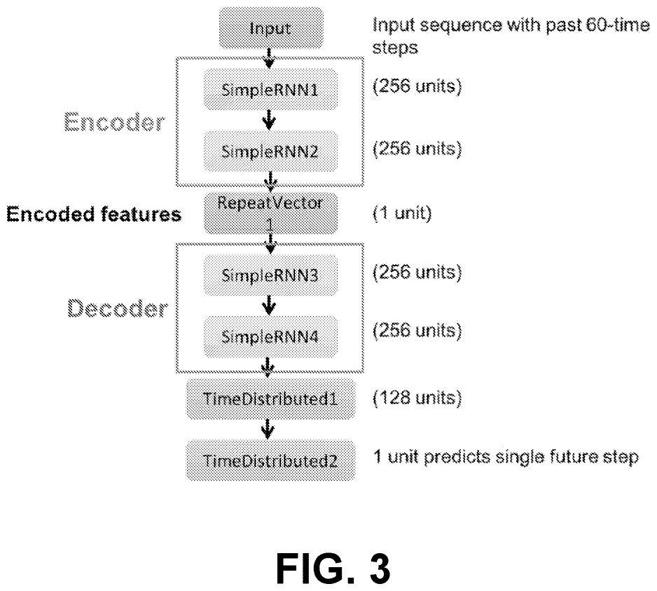

FIG. 3 shows a generic autoencoder predictor architecture with SimpleRNN layers. The SimpleRNN Autoencoder has an input sequence of one feature for 60 time steps. The SimpleRNN1 layer with 256 units reads the input data and provides the output with 256 features. The SimpleRNN2 layer with 256 units takes the previous output of SimpleRNN1 layer as an input and generates the output with 256 features as an encoded feature vector. The RepeatVector layer, with one unit, repeats the feature vector one time. In general, Decoder module layers are put into reverse order of the Encoder module for decoding the encoded information. The SimpleRNN3 layer is identical to the SimpleRNN2 layer, and the SimpleRNN4 layer is identical to the SimpleRNN1 layer. The TimeDistributed1 layer with 128 units is added to reduce the 256 features to 128 features. The last TimeDistributed2 layer with 1 unit is added to receive the predicted output at one-step ahead.

The hyper-parameters of SimpleRNN Autoencoder were selected based on the minimum validation loss criteria. Batch size of 50,000, ADAM optimizer with initial learning rate of 0.01, ReLU activation function, and 200 epochs were used for training this NN architecture. Keras ReduceLROnPlateau callback was used to handle the learning saturation issue when a validation loss did not show improvement. The learning rate was reduced by half if there was no improvement on validation loss over 10 continuous epochs. The model weights with the best minimum validation loss were applied to make predictions on test input sequences of heart rates.

### LSTM Autoencoder Predictor

The LSTM neural networks solve the issue of vanishing and exploding gradients and are capable of learning long term dependencies by remembering in formation for a long time period. The LSTM unit is a composition of cells and three gates. These gates (forget, input, and output) control the flow of information into and out of the cell. The cell stores the information in memory for a longer duration. At each time step t, the hidden state and new cell state are updated, and the gates of LSTM units make decisions on which states to add or remove.

LSTM networks incorporated into Encoder-Decoder architecture were tested to provide flexibility in supporting the variable length of the input sequence and the variable length of the output sequence. The architecture of the LSTM Encoder predictor is similar to that of the SimpleRNN shown in FIG. 3 where SimpleRNN cells were replaced with LSTM. The hyper-parameters of LSTM Autoencoder predictors are similar to the hyper-parameters of SimpleRNN Autoencoder predictors.

### Bi-LSTM Autoencoder Predictor

The unidirectional LSTM takes an input and preserves only the past states information. The Bi-LSTM is an extension of LSTM that consists of two LSTM models. The study discusses architectural differences between LSTM and Bi-LSTM and compares the performance of both models on predicting stock data. The Bi-LSTM network processes the given inputs in forward and backward directions (using Forward LSTM and Backward LSTM): (1) past to future states, (2) future to past states. These two layers are combined together to provide the information in both directions to improve the long-term dependencies and accuracy of the model.

The architecture of the Bi-LSTM autoencoder predictors is similar to the SimpleRNN shown in FIG. 3 with SimpleRNN cells replaced with Bi-LSTM. The hyper-parameters of Bi-LSTM Autoencoder predictors are similar to the hyper-parameters of SimpleRNN Autoencoder predictors.

### GRU Autoencoder Predictor

The Gated Recurrent Unit (GRU) is a type of RNN and a variant of LSTM with a different gating mechanism. The parameters of LSTM are reduced by the design of GRU to improve the performance and make the architecture more simplistic. Thus, GRU trains faster compared to LSTM due to having fewer parameters. As discussed earlier, LSTM's architecture has three gates (forget, input, and output), whereas, the GRU has only two gates (update and reset). The update gate combines the forget gate and input gate of the LSTM. It decides on the amount of prior information (from past time steps) needed to be passed along to the next state. The reset gate decides on the amount of past information needed to be discarded.

The GRU autoencoder predictor architecture is similar to that of the Sim-pleRNN Autoencoder predictor shown in FIG. 3, except for the difference in memory cell usage. The hyper-parameters of GRU Autoencoder predictors are similar to the hyper-parameters of other autoencoder predictors.

### Transformer Predictor

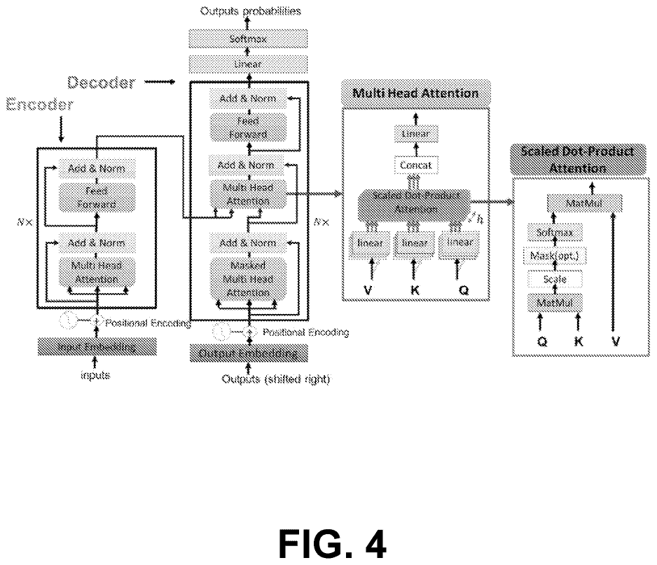

The Transformer is a deep learning model designed to handle sequential data using Encoder and Decoder components based on attention mechanisms. Transformer, unlike RNN-based models, does not process data in an ordered sequence. Instead, it processes the entire sequence of data and uses the attention mechanism to learn complex patterns and dynamics from time series data. Each attention processor of a Transformer is called the attention head and is repeated several times in parallel to propose the Multi-head attention. The Multi-head attention provides the capability to extract the information from different representation sub-spaces instead of a single attention head. The Encoder reads the input sequence and maps it into a feature vector of a higher dimension. The Decoder module then uses this feature vector to transform it into an output sequence. The Encoder and Decoder modules of the Transformer are shown on the left and right side in FIG. 4. The attention mechanism is implemented to focus on the most important subset of long input sequences. These modules can be stacked multiple times (shown with N×symbol on FIG. 4) while proposing the design of a Transformer.

In this work, the heart rate was predicted five seconds ahead by implementing the Transformer architecture presented in and using the code given in GitHub repositories.

### Statistics-Based Predictors:

We propose to compare the Last Observation Carried Forward (LOCF), Linear Regression, Linear, and Finite Difference predictors with the listed above state-of-the-art DL-based predictors. Note that the statistical predictors do not require any training and are therefore very simple to implement on any hardware.

### LOCF Predictor

LOCF is a data imputation technique where the missing values are replaced with the previously observed value from the data. The method used for predicting the signal:

$$\hat{h} ( t + T ) = h ( t ) \qquad ( 2 )$$

For T=5 seconds, the LOCF predictor takes the five seconds ago value of a heart rate as the present predicted value.

### Linear Regression Predictor

Linear regression is commonly used in linear predictive modeling techniques and in fitting a cloud of points to a straight-line model. The output of the prediction can be calculated using the linear combination of the previous input features with linear least squares error fit of a function. The proposed Linear Regression predictor uses four samples from the past and present (h(t), h(t-1), h(t-2), and h(t-3)) to fit a straight-line model and extrapolate it for t+T time moment, where T=5. The fitted model could be obtained analytically, and the predictor is given by:

$$\hat{h} ( t + T ) = 2.2 h ( t ) + 0.9 h ( t - 1 ) - 0.4 h ( t - 2 ) - 1.7 h ( t - 3 ) . \qquad ( 3 )$$

### Finite Difference Predictor

In this section a predictor based on finite differences was implemented. The assumption for this predictor is that the approximation of a second order derivative is a constant. The predictor uses four signal samples from the last four time steps h(t), h(t-1), h(t-2), and h(t-3) to predict the 5th step (second):

$$\hat{h} ( t + T ) = 13.5 h ( t ) - 12.5 h ( t - 1 ) - 7.5 h ( t - 2 ) - 7.5 h ( t - 3 ) . \qquad ( 4 )$$

This equation is obtained by computing the first backward difference: h(t-2)−h(t-3), h(t-1)−h(t-2), h(t)−h(t-1), and then the second backward difference h(t-1)−2h (t-2)+h(t-3), h(t)−2h (t-1)+h(t-2). In general, the two obtained values are different but average is computed m=0.5 (h(t)−h(t-1)−h(t-2)+h(t-3)) and it is assumed that the same number should continue for T=5 more steps. The first order difference is extrapolated by repetitive additions of m and the original sequence is extrapolated by repetitive addition of the extrapolated first differences. The 5th extrapolated value is given by the equation (4).

### Linear Predictor

The linear predictor uses the past two samples h(t) and h(t-1) to predict the fifth time step, assuming that the slope of a signal will stay constant:

$$\hat{h} ( t + T ) = h ( t ) + 5 ( h ( t ) - h ( t - 1 ) ) = 6 h ( t ) - 5 h ( t - 1 ) . \qquad ( 5 )$$

### Evaluation of Event Predictors

In the present section, new definitions are provided for the statistical metrics of Sensitivity, Specificity, Precision, Accuracy, and F1 score according to the scenario with prolonged events and potential clinical interventions in real time.

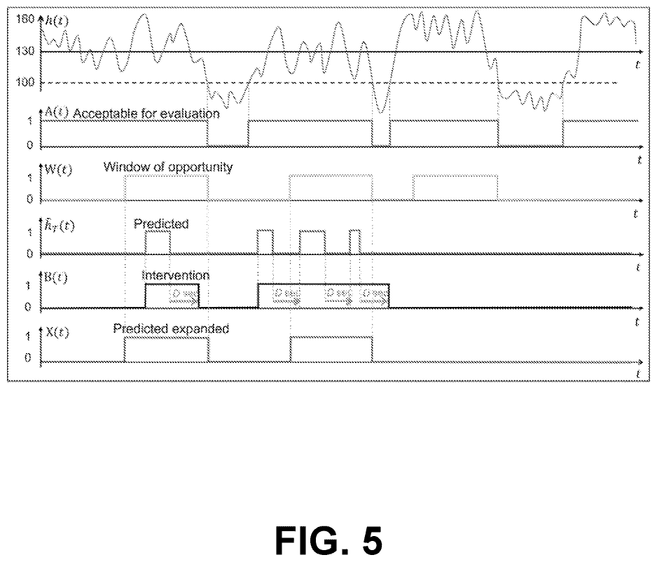

Let a biomedical signal h: [symbol] → [symbol] be a function of time t=1, 2, . . . , N, and define the T-seconds ahead predictor binary output (see FIG. 5) by:

$${\hat{h}}_{T} ( t ) = \{ \begin{matrix}0 , if \hat{h} ( t ) < τ \\ 1 , otherwise\end{matrix} , \qquad ( 6 )$$

where 0 means that the event is not predicted, and 1 means that the event is predicted. The τ is an arbitrary predefined threshold for deciding if some predicted value ĥ_{T}(t) is marking an event. Note that for high values of τ the predictor is much less likely to detect any events since it will almost always be at 0, but it is also not likely to produce many false alarms. If τ is very low, then the situation is opposite and the predictor will detect more true events, but is likely to produce more false alarms. Clearly the choice of τ will affect the accuracy, precision, and other evaluation measures, but it adds flexibility and allows selection of the τ value to achieve the desired characteristics.

A predictor denoted by ĥ_{T}(t) is computing its prediction based on h(t) in the past time interval [t-T-w, t-T], where w∈N is the size of a constant sliding 1D window. Given h(t) and ĥ_{T}(t), the predictor's efficiency can be evaluated.

Generally acceptable definitions for true positive (TP), true negative (TN), false positive (FP), and false negative (FN) as follows:
TP—is the number of cases, where an event has been predicted and the event really occurred;
FP—is the number of cases, where an event has been predicted but the event did not occur;
TN—is the number of cases, where an event has not been predicted and the event did not occur;
FN—is the number of cases, where an event has not been predicted but an event occurred.

To describe the evaluation metrics in terms of successful event prediction, the following Boolean functions are introduced (see FIG. 5).

The function A: [symbol] →{0, 1} is defined for t∈[1, N] such that:

$$A ( t ) = \{ \begin{matrix}0 , if h ( t ) < 100 \\ 1 , otherwise\end{matrix} , \qquad ( 7 )$$

The function A is 1 when there is no bradycardia (FIG. 5). Note, the condition h(t)<100 could be replaced with any other.

The function W: [symbol] →{0, 1} is the window of opportunity to predict the event. For the particular case of bradycardia prediction, in one minute of vital signs recording prior to an event time window (60 samples) is of interest because in that minute biological systems start to lose stability. For theoretical purpose, the interval is generalized to Δt∈N. The set of all event onset time samples is defined as e={t₁, t₂, . . . , tₙ}, where n is the number of bradycardia events within the 24 hours of recording. The function W(t) is defined below and illustrated in FIG. 5:

$$W ( t ) = \{ \begin{matrix}0 , if t ∈ [ {t}_{i} - Δ t , {t}_{i} - 1 ] ; ∀ i = 1 , 2 , … , n \\ 1 , otherwise\end{matrix} . \qquad ( 8 )$$

Now, it is assumed that there might be an intervention following the prediction of an event. This intervention, whether it is a buzzer to wake up the infant and restore their heart rate, or any other real-time intervention, is modifying the original signal; thus, no additional prediction is necessary within this interval. The Boolean function B: [symbol] →{0, 1} is 1 when the buzzer (or intervention) is on. The buzzer starts to work when the predictor shows value 1 (∃t: ĥ_{T}(t)=1) and stays on for D more samples (seconds) after the predictor dropped to 0 values (ĥ_{T}(t)=0). Formally, the predicted signal ĥ_{T}(t) can be expanded using morphological dilation of D seconds to the right as follows:

$$B ( t ) = {\hat{h}}_{T} ( t ) ⊕ V = {⋃}_{i ∈ V} {\hat{h}}_{T} ( t - i ) \qquad ( 9 )$$

where V=(0, 1, 2 . . . , D) is a structuring element vector. In other words, B(t) is the union of ĥ_{T}(t), ĥ_{T}(t-1), ĥ_{T}(t-2), ĥ_{T}(t-D) binary functions, where ĥ_{T}(t-1) denotes a shift of the function ĥ_{T}(t) a step to the right. Union of Boolean functions is defined as ĥ_{T}(t-k)∪ĥ_{T}(t-j)=1 if and only if ĥ_{T}(t-k)=1 or ĥ_{T}(t-j)=1. Otherwise ĥ_{T}(t-k)∪ĥ_{T}(t-j)=0.

The expanded prediction Boolean function X: [symbol] →{0, 1} is 1 everywhere within the window of opportunity W=1, where the predictor ĥ_{T}(t)=1 (predicted event) at least once. The reason to define such a function is because if a predictor has predicted the event correctly anywhere within the window of opportunity W(t), it is counted as a successful event detection regardless of how long and how many times it was predicted within this window. The function X(t) is defined using morphological reconstruction with ĥ_{T}(t) marker and W(t) mask. The iterative algorithm for the computation of X(t) is given as following:
1. X₁(t)←ĥ_{T}(t)
2. Repeat Xₖ₊₁(t)←W(t)∩(Xₖ(t)⊕(1,1,1)) until ∀t: Xₖ₊₁(t)=Xₖ(t).
3. The reconstructed function X(t)=Xₖ₊₁(t).

Using the above Boolean functions together with the basic logical operations AND (logical multiplication) and NEGATE the following novel definitions of the well-known metrics are formulated:

$$T P = \frac{{\sum }_{t = 1}^{N} A ( t ) W ( t ) X ( t )}{{\sum }_{t = 1}^{N} A ( t )} , \qquad ( 10 )$$

$$FP = \frac{{\sum }_{t = 1}^{N} A ( t ) B ( t ) \overline{W} ( t )}{{\sum }_{t = 1}^{N} A ( t )} , \qquad ( 11 )$$

$$TN = \frac{{\sum }_{t = 1}^{N} A ( t ) \overline{B} ( t ) \overline{W} ( t )}{{\sum }_{t = 1}^{N} A ( t )} , \qquad ( 12 )$$

$$FN = \frac{{\sum }_{t = 1}^{N} A ( t ) W ( t ) \overline{X} ( t )}{{\sum }_{t = 1}^{N} A ( t )} , \qquad ( 13 )$$

where the bar above a Boolean function denotes logical negation. In addition, the true prediction (T Pred) notation is defined for events predicted within the windows of opportunity.

$$T Pred = \frac{{\sum }_{t = 1}^{N} X ( t )}{{\sum }_{t = 1}^{N} W ( t )} , \qquad ( 14 )$$

Note that false alarm (FA) and false positive (FP) are used interchangeably, since they have the same meaning in the context herein.

### Lemma 3.1. TP+FP+TN+FN=1

### Proof:

$$TP + FP + TN + FN = \frac{{\sum }_{t = 1}^{N} AWX}{{\sum }_{t = 1}^{N} A} + \frac{{\sum }_{t = 1}^{N} AB \overline{W}}{{\sum }_{t = 1}^{N} A} + \frac{{\sum }_{t = 1}^{N} A \overline{B} \overline{W}}{{\sum }_{t = 1}^{N} A} + \frac{{\sum }_{t = 1}^{N} AW \overline{X}}{{\sum }_{t = 1}^{N} A} = \frac{{\sum }_{t = 1}^{N} ( AWX + AB \overline{W} + A \overline{B} \overline{W} + AW \overline{X} )}{{\sum }_{t = 1}^{N} A} = \frac{{\sum }_{t = 1}^{N} A ( W ( X + \overline{X} ) + ( B + \overline{B} ) \overline{W} )}{{\sum }_{t = 1}^{N} A} = \frac{{\sum }_{t = 1}^{N} A W}{{\sum }_{t = 1}^{N} A} = 1 \qquad ( 15 )$$

Based on TP, FP, TN and FN, the model efficiency can be evaluated by using Sensitivity/Recall, Specificity, Precision, Accuracy, and F1score metrics:

$$Sensitivity = \frac{T P}{T P + F N} , \qquad ( 16 )$$

$$Specificity = \frac{T P}{T N + F P} , \qquad ( 17 )$$

$$Precision = \frac{T P}{T P + E P} , \qquad ( 18 )$$

$$Accuracy = \frac{T N + T P}{T N + F P + F N + T P} , \qquad ( 19 )$$

$${F}_{1} = \frac{2 ( Sensitivity * Precision )}{Sensitivity + P r e c i s i o n} , \qquad ( 20 )$$

### Results and Discussion

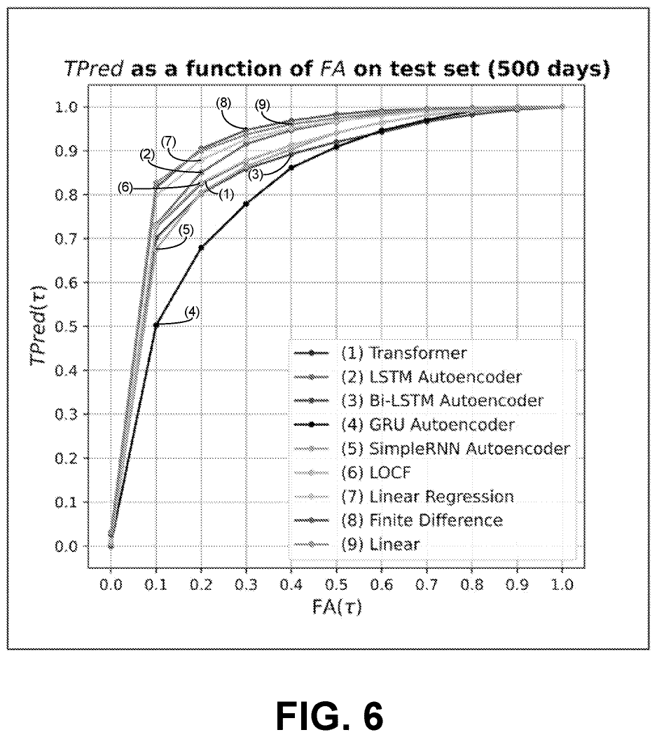

In the experiments a sliding window of w=60 seconds is used to test predictors. FIG. 6 shows the performance comparison of nine 5-steps ahead predictors (the five DL-based predictors and four statistics-based predictors described) on a testing set using false alarm (FA) and true predicted (TPred) measures.

Each of these measures is a function of τ, and an average parametric plot for T_{Pred}(τ) is shown as a function of (FA(τ). Note that each predictor is shown on a different τ scale. Since τ has an arbitrary range, its absolute value does not matter as long as for a specific τ value one gets acceptable predictions.

The parameter τ∈[100, +100] range was selected for the following predictors: Transformer, LSTM Autoencoder, Bi-LSTM Autoencoder, GRU Autoencoder, SimpleRNN Autoencoder, and LOCF. The range of τ was increased for Linear Regression, Linear, and Finite Difference predictors in order to cover all the possible values of Sensitivity, Precision, and F1 score. The τ∈[−200, +200] range was selected for a Linear Regression predictor, τ∈[−400, +400] range was selected for a Linear model, and τ∈[−1000, +1000] was selected for a Finite Difference predictor. The intervention length of D=15 seconds is used.

An important part of the plots is on the left (with lower FA values), and for a working point only a single point is chosen from the plot with an acceptable FA. For a given FA rate, the highest possible TPred is determined. While most DL-based predictors perform reasonably well in that range, Linear and finite difference models outperform all state-of-the-art artificial neural networks and predict correctly more events per given FA rate. Table 2 summarizes the mean TPred value for the chosen FA rate of 0.1.

Table 2 summarizes the mean TPred value for the chosen FA rate of 0.1 (10%). The best result is obtained by the simplest linear predictor while com-pared on 500 days of recording. The second best for FA<0.1 is the Finite difference predictor. The performances of all DL-based methods is comparable with the LOCF predictor for the testing and about 10% worse than the other simple predictors. Nevertheless, adding more training data in the future might improve predictions made by DL algorithms.

TABLE 2
Prediction performance comparison of nine different predictors
using mean and standard deviation (SD) values. The second
column mean represent the mean value for results of TPred
at (F A = 0.1). The third column (SD) represents
the standard deviation values for each predictor, respectively.
The three best predictors are in bold.

| Predictor | mean | SD |
|---|---|---|
| (1) Transformer | 0.73 | 0.21 |
| (2) LSTM Autoencoder | 0.73 | 0.24 |
| (3) Bi-LSTM Autoencoder | 0.70 | 0.26 |
| (4) GRU Autoencoder | 0.50 | 0.27 |
| (5) SimpleRNN Autoencoder | 0.68 | 0.25 |
| (6) LOCF | 0.73 | 0.21 |
| (7) Linear Regression | 0.80 | 0.20 |
| (8) Finite Difference | 0.82 | 0.20 |
| (9) Linear | 0.83 | 0.19 |

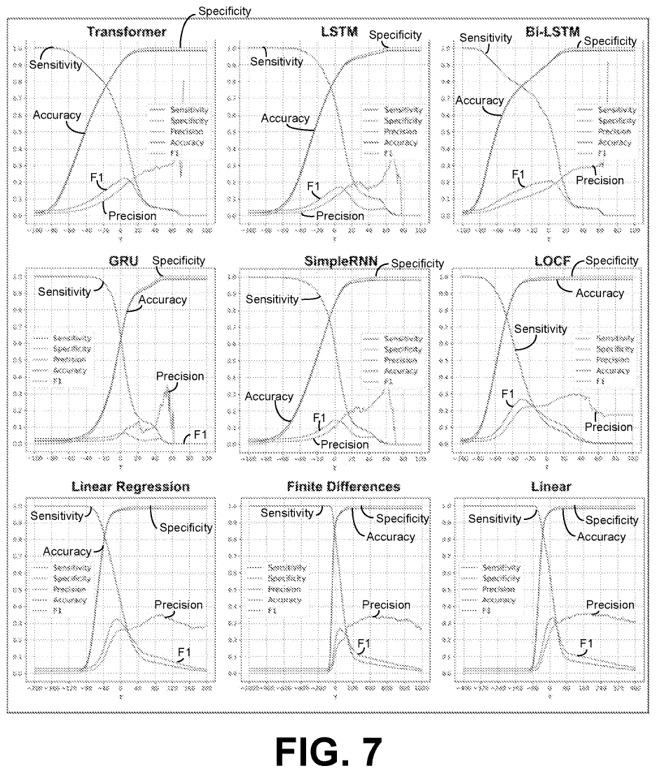

FIG. 7 shows an average sensitivity, specificity, precision, accuracy, and F1 score calculated with the help of the newly developed measures for the testing set. Based on these metrics, the simplest Linear and Finite Differences predictors provide better results than DL-based techniques.

### SUMMARY

A few simple predictors show superior results with biomedical signals. The proposed evaluation methods allow accounting for prolonged events (where event duration matters) and potential real-time clinical interventions affecting the signals. The developed metrics were verified through nine DL and statistics-based predictors with regard to their efficiency in successful prediction of bradycardia events in preterm infants. Clearly, the mathematical formalism of bradycardia events predictions is adaptable to prediction of other medical events. The proposed metrics could be applied to measure the effectiveness of prediction of other clinically significant events such as desaturations, epileptic seizures, asthma attacks, or cardiac events.

Results, both from the Table 2 and FIG. 6, show that Finite Difference and Linear predictors have achieved the best average performance. While DL-based approaches are used with high level of success in the image analysis domain, 1D signal analysis and prediction still pose many challenges for them. More data could be gathered to obtain better training for the DL-based predictors. This would handle the data variability of the training set and would diminish the problem of unbalanced training with data sets where the events are rare. The methods were developed for univariate time series prediction, but can be expanded to multi-variable prediction problems as well.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’ includes “about ‘x’ to about ‘y’”.

We claim:

1. A system, comprising:
at least one computing device in data communication with at least one sensor positioned on a person being monitored;
an application executable in the at least one computing device, wherein when executed, the application causes the at least one computing device to at least:
receive biomedical signal information from the at least one sensor positioned on the person, the biomedical signal information comprising at least heart rate data;
evaluate a sequence of heart rate values over an interval of time based at least in part on the heart rate data;
forecast a predicted heart rate value for a subsequent time;
in response to the predicted heart rate value being less than a predetermined threshold value, generate an intervention signal to prompt an intervention; and
transmit the intervention signal to an intervention apparatus in communication with the at least one computing device, the intervention apparatus adapted to receive the intervention signal from the at least one computing device and cause at least a vibration to simulate the person.

2. The system of claim 1, wherein the intervention apparatus comprises a wearable vibrotactile device positioned on the person being monitored.

3. The system of claim 1, wherein the intervention apparatus comprises a vibrotactile device is positioned in or under a bed in which the person being monitored is located.

4. The system of claim 1, wherein forecasting the predicted heart rate value for the subsequent time interval comprises calculating a linear algorithm.

5. The system of claim 1, wherein forecasting the predicted heart rate value for the subsequent time interval comprises calculating a linear regression algorithm.

6. The system of claim 1, wherein forecasting the predicted heart rate value for the subsequent time interval comprises calculating a finite difference algorithm.

7. The system of claim 1, wherein, when executed, the application further causes the at least one computing device to at least generate an additional alert in response to the predicted heart rate value being less than the predetermined threshold value.

8. The system of claim 7, wherein the additional alert is at least one of: an audible alert and a visual alert.

9. The system of claim 7, wherein the additional alert comprises a notification to an external system.

10. The system of claim 7, wherein the at least one computing device is in communication with a smart device configured to communicate with a third party, and generating the additional alert further comprises instructing the smart device to send a communication with the third party.

11. A method for predicting and interrupting bradycardia episodes in a person, comprising:
receiving, via at least one computing device, biomedical signal information comprising at least heart rate data of the person being monitored;
evaluating, via the at least one computing device, a sequence of heart rate values over an interval of time based at least in part on the heart rate data;
forecasting, via the at least one computing device, a predicted heart rate value for a subsequent time;
in response to the predicted heart rate value being less than a predetermined threshold value, generating, via the at least one computing device, an intervention signal to prompt intervention; and
transmitting the intervention signal to an intervention apparatus in communication with the at least one computing device, the intervention apparatus adapted to receive the intervention signal from the at least one computing device and cause at least a vibration to simulate the person.

12. The method of claim 11, wherein the intervention apparatus comprises a wearable vibrotactile device positioned on the person being monitored.

13. The method of claim 11, wherein the intervention apparatus comprises a vibrotactile device is positioned in or under a bed in which the person being monitored is located.

14. The method of claim 11, wherein forecasting the predicted heart rate value for the subsequent time interval comprises calculating a linear algorithm.

15. The method of claim 11, wherein forecasting the predicted heart rate value for the subsequent time interval comprises calculating a linear regression algorithm.

16. The method of claim 11, wherein forecasting the predicted heart rate value for the subsequent time interval comprises calculating a finite difference algorithm.

17. The method of claim 11, further comprising generating, via the at least one computer device, an additional alert in response to the predicted heart rate value being less than the predetermined threshold value.

18. The method of claim 17, wherein the additional alert is at least one of: an audible alert and a visual alert.

19. The method of claim 17, wherein the additional alert comprises a notification to an external system.

20. The method of claim 17, wherein the at least one computing device is in communication with a smart device configured to communicate with a third party, and generating the additional alert further comprises instructing the smart device to send a communication with the third party.
